(19) United States
(12) Patent Application Publication
(10) Pub. No.: US 2026/0212971 A1
(43) Pub. Date: Jul. 23, 2026

(54) SYSTEM, APPARATUS, AND METHOD FOR DONATION MANAGEMENT

(71) Applicant: Bikram Jit SINGH, Chandigarh (IN)

(72) Inventor: Bikram Jit SINGH, Chandigarh (IN)

(21) Appl. No.: 19/142,648

(22) PCT Filed: Dec. 5, 2023
(86) PCT No.: PCT/IN2023/051135

§ 371 Date: Jun. 24, 2025

(30) Foreign Application Priority Data
Dec. 24, 2022 (IN) 202211075186

(51) Int. Cl. G16H 10/40; G06Q 50/26

(52) U.S. Cl. CPC G16H 10/40; G06Q 50/26

(57) ABSTRACT

Disclosed data processing apparatus (104) includes a processing circuitry (120) that generates a user identifier (UID) for each user of a plurality of users based on a set of user inputs, generates a sample identifier (SID) for each donation sample of the one or more donation samples donated by a first user of the plurality of users, and associates the SID of each donation sample with a UID of the first user upon a donation of a donation sample and a UID of a second user of the plurality of users upon a reception of the donation sample. The processing circuitry (120) further distributes the first and second user profiles on a plurality of data storage nodes (106), and sets priorities of the first and second users for donation and reception of one or more donation samples based on the first and second user profiles, respectively.

### TECHNICAL FIELD

The present disclosure relates to the technical field of medical management systems. More particularly, the present disclosure relates to a system, an apparatus, and a method for donation management.

### BACKGROUND

Medical emergencies, accidents, and fatalities impact a sufferer and its family for a long time and can result in a trauma for lifetime. Severe medical emergencies can require blood or other organs to be provided/transplanted to save life of the sufferer in a very specific span of time. A good percentage of people facing severe medical emergencies lose their lives due to unavailability of blood, blood components, or organs in the required time.

Some of the major reasons for the abovementioned situation is lack of awareness amongst population, illegal sales (black marketing) of blood and organs, and absence of any regulatory authority (or system) that can provide real-time monitoring in order to control the inventory wastage and unlicensed donation centers. Specifically, the wastage of blood units and donated organs occur due to different lifespans of different components of the blood. For example, upon donation and when kept under predefined environmental conditions, whole blood can be used for 21-35 days, red blood cells can be used up to 42 days, platelets can be used for five days, and plasma can be used up to one year. Similarly, different body organs also have different lifespans by which they should be transplanted. Thus, it becomes critical to manage each donation sample according to their respective lifespans to minimize wastage.

Every adult human being (male or female) has 5 to 6 liters of blood. Any healthy person between 18 and 60 years of age and weighing 45 kg or more can safely donate one unit of blood (i.e., 350 ml) once in every three months. Similarly, a part of liver can be donated by a healthy person to one in need. There is a need to motivate and educate people about blood and organ donation, and regulate and manage the donations to keep up to the requirements of blood and organs.

Thus, a secure system with automated donation management, that is capable of providing real-time management of healthcare donations is an ongoing effort and demands a need for improvised technical solution that overcomes the aforementioned problems.

### SUMMARY

In an aspect of the present disclosure, a data processing apparatus includes processing circuitry. The processing circuitry is configured to generate, based on a set of user inputs received from a user device, a user identifier (UID) for each user of a plurality of users, that is associated with a donation of one or more donation samples such that the one or more donation samples comprising at least one of, one or more blood units, one or more body organs, or a combination thereof. The processing circuitry is further configured to generate a sample identifier (SID) for each donation sample of the one or more donation samples donated by a first user of the plurality of users. Furthermore, the processing circuitry is configured to associate the SID of each donation sample with a UID of the first user upon a donation of the donation sample and a UID of a second user of the plurality of users upon a reception of the donation sample. Furthermore, the processing circuitry is configured to generate first and second user profiles for the first and second users, respectively, based on the association. Furthermore, the processing circuitry is configured to distribute the first and second user profiles on a plurality of data storage nodes. Furthermore, the processing circuitry is configured to set a priority of the first and second users for donation and reception of one or more donation samples based on the first and second user profiles, respectively.

In some aspects, to generate the first and second user profiles, the processing circuitry is configured to determine a count of donation samples donated and received by the first and second users, respectively, based on a count of SIDs associated with the UID of the first and second users, respectively. The processing circuitry is further configured to generate first and second donation scores for the first and second users based on the count of donation samples donated and received by the first and second users, respectively. Furthermore, the processing circuitry is configured to generate, first and second status flag for the first and second users, respectively, based on a set of health inputs of the first and second users received from the user device and the first and second donation scores. The first user profile includes a UID of the first user, the first donation score, and the first status flag, and the second user profile comprising a UID of the second user, the second donation score, and the second status flag, respectively. In some aspects, the processing circuitry is further configured to associate the SIDs of the at least one donation sample with the UID of a third user of the plurality of users that collects at least one donation sample of the one or more donation sample from the first user and provides the at least one donation sample to the second user. Furthermore, the processing circuitry is configured to determine a count of donation samples of the at least one donation sample collected or provided by the third user, based on a count of SIDs associated with the UID of the third user. Furthermore, the processing circuitry is configured to generate a third donation score for the third user based on the count of donation samples collected or provided by the third user. Furthermore, the processing circuitry is configured to generate a third status flag for the third user based on the set of health inputs of the first and second users received from the user device and the third donation score. The processing circuitry is configured to generate a third user profile for the third user based on the third status flag, the third UID and the third donation score. Furthermore, the processing circuitry is configured to distribute the third user profile on the plurality of data storage nodes, and set a priority of the third user for at least one of, the donation of one or more donation samples from the first user and the reception of one or more donation samples from the second user based on the third user profile.

In some aspects, the processing circuitry is further configured to generate a goods identifier (GID) of each health good of the one or more health goods provided by a fourth user that provides one or more health goods to at least one of, the first through third users, or a combination thereof. Furthermore, the processing circuitry is configured to associate the GIDs of the one or more health goods with a UID of the fourth user. Furthermore, the processing circuitry is configured to determine a first through third count of goods provided by the fourth user to the first through third users based on a count of GIDs associated with the UID of the first through third users, respectively. Furthermore, the processing circuitry is configured to generate a sale score for the fourth user based on first through third count of goods. Furthermore, the processing circuitry is configured to generate a fourth status flag for the fourth user based on the sale score. Furthermore, the processing circuitry is configured to generate a fourth user profile for the fourth user based on the fourth status flag, the fourth UID and the sale score, and distribute the fourth user profile on the plurality of data storage nodes.

In another aspect of the present disclosure, a method includes generating, by way of processing circuitry, a user identifier (UID) for each user of a plurality of users, that is associated with a donation of one or more donation samples, based on a set of user inputs received from a user device. The one or more donation samples comprising at least one of, one or more blood units, one or more body organs, or a combination thereof. The method further includes generating, by way of the processing circuitry, a sample identifier (SID) for each donation sample of the one or more donation samples donated by a first user of the plurality of users. Furthermore, the method includes associating, by way of the processing circuitry, the SID of each donation sample with a UID of the first user upon a donation of the donation sample and a UID of a second user of the plurality of users upon a reception of the donation sample. Furthermore, the method includes generating, by way of the processing circuitry, first and second user profiles for the first and second users, respectively, based on the association. Furthermore, the method includes distributing, by way of the processing circuitry, the first and second user profiles on a plurality of data storage nodes. Furthermore, the method includes setting, by way of the processing circuitry, a priority of the first and second users for donation and reception of one or more donation samples based on the first and second user profiles, respectively.

### BRIEF DESCRIPTION OF DRAWINGS

The above and still further features and advantages of aspects of the present disclosure becomes apparent upon consideration of the following detailed description of aspects thereof, especially when taken in conjunction with the accompanying drawings, and wherein:

FIG. 1 illustrates a block diagram of a system for donation management, in accordance with an exemplary aspect of the present disclosure;

FIG. 2 illustrates a data processing apparatus of the system of FIG. 1, in accordance with exemplary aspect of the present disclosure; and

FIG. 3 illustrates a flow diagram of a method for donation management, in accordance with an aspect of the present disclosure.

To facilitate understanding, reference numerals have been used, where possible, to designate elements common to the figures.

### DETAILED DESCRIPTION

Various aspects of the present disclosure provide a system and a method for donation management. The following description provides specific details of certain aspects of the disclosure illustrated in the drawings to provide a thorough understanding of those aspects. It should be recognized, however, that the present disclosure can be reflected in additional aspects and the disclosure may be practiced without some of the details in the following description.

The various aspects including the example aspects are now described more fully with reference to the accompanying drawings, in which the various aspects of the disclosure are shown. The disclosure may, however, be embodied in different forms and should not be construed as limited to the aspects set forth herein. Rather, these aspects are provided so that this disclosure is thorough and complete, and fully conveys the scope of the disclosure to those skilled in the art. In the drawings, the sizes of components may be exaggerated for clarity.

It is understood that when an element is referred to as being “on,” “connected to,” or “coupled to” another element, it can be directly on, connected to, or coupled to the other element or intervening elements that may be present. As used herein, the term “and/or” includes any and all combinations of one or more of the associated listed items.

The subject matter of example aspects, as disclosed herein, is described with specificity to meet statutory requirements. However, the description itself is not intended to limit the scope of this patent. Rather, the inventor/inventors have contemplated that the claimed subject matter might also be embodied in other ways, to include different features or combinations of features similar to the ones described in this document, in conjunction with other technologies. Generally, the various aspects including the example aspects relate to the system, and the method for donation management. Specifically, the various aspects of the present disclosure relate to management of donations related to healthcare such as blood donation, organ donation, and the like.

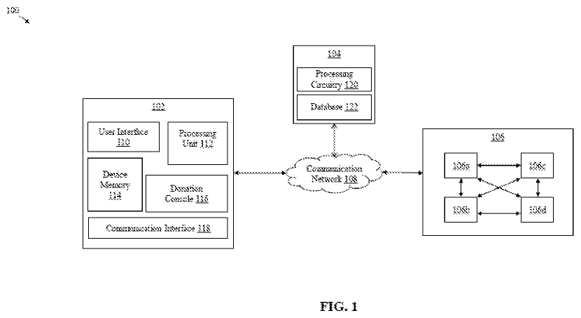

FIG. 1 illustrates a block diagram of a system 100 for donation management, in accordance with an exemplary aspect of the present disclosure.

In some aspects of the present disclosure, the system 100 may provide a distributed platform for management of healthcare donation in the form of a web version and an application version. The system 100 may further facilitate a plurality of users to communicate with each other via Communication Platform as a Service (CPaaS) via an external third party application.

In some aspects of the present disclosure, the system 100 may be configured send notifications regarding one or more upcoming donation camps to the plurality of users. In some aspects of the present disclosure, the system 100 may be configured to enable the plurality of users to retrieve individual geographical locations via an external third party application. The system 100 may further enable the plurality of users to share the individual geographical locations (via Bluetooth or internet) to determine the proximity with respect to each other which may enable the plurality of users to locate the donation camp. In some aspects of the present disclosure, the system 100 may further be configured to obtain an estimated time of travel and an estimated time of arrival at a selected donation camp.

In some aspects of the present disclosure, the system 100 may generate a notification code or a one-time password (OTP) to a first user (i.e., a donor) of plurality of users to submit a consent of donation.

The system 100 may include a user device 102, a data processing apparatus 104 and a plurality of data storage nodes 106. In some aspects of the present disclosure, the user device 102 and the plurality of data storage nodes 106 may be communicatively coupled to the data processing apparatus 106, by way of a communication network 108. In some aspects of the present disclosure, the communication network 108 may provide wired or wireless communication medium (e.g., communication channels) to enable sharing data and instructions between the user device 102, the plurality of data storage nodes 106 and the data processing apparatus 106.

In some aspects of the present disclosure, the user device 102 may act as a medium to access the data processing apparatus 104 and the plurality of data storage nodes 106 for the plurality of users. The user device 102 may include a user interface 110, a processing unit 112, a device memory 114, a donation console 116, and a communication interface 118.

The user interface 110 may include an input interface for receiving inputs from a user of the plurality of users associated with one or more situations related to healthcare such as, but not limited to, selection of a user category, registration of a user, login of a user, donation and/or reception of a sample by a user, sale and/or purchase of healthcare goods and supplements by a user, notification of an emergency generated by a user, and the like. Examples of the input interface may include, but are not limited to, a touch interface, a mouse, a keyboard, a motion recognition unit, a gesture recognition unit, a voice recognition unit, or the like. Aspects of the present disclosure are intended to include or otherwise cover any type of the input interface including known, related art, and/or later developed technologies without deviating from the scope of the present disclosure. The user interface 110 may further include an output interface for displaying (or presenting) outputs to a user of the plurality of users. Examples of the output interface of the user interface 110 may include, but are not limited to, a digital display, an analog display, a touch screen display, a graphical user interface, a website, a webpage, a keyboard, a mouse, a light pen, an appearance of a desktop, and/or illuminated characters.

The processing unit 112 may include suitable logic, instructions, circuitry, interfaces, and/or codes for executing various operations, such as the operations associated with the user device 102, or the like. In some aspects of the present disclosure, the processing unit 112 may utilize one or more processors such as Arduino or raspberry pi or the like. Further, the processing unit 112 may be configured to control one or more operations executed by the user device 102 in response to the input received at the user interface 110 from the user. Examples of the processing unit 112 may include, but are not limited to, an application-specific integrated circuit (ASIC) processor, a reduced instruction set computing (RISC) processor, a complex instruction set computing (CISC) processor, a field-programmable gate array (FPGA), a Programmable Logic Control unit (PLC), and the like. Some aspects of the present disclosure are intended to include or otherwise cover any type of the processing unit 110 including known, related art, and/or later developed processing units.

The device memory 114 may be configured to store the logic, instructions, circuitry, interfaces, and/or codes of the processing unit 112, data associated with the user device 102, and data associated with the system 100. Examples of the device memory 114 may include, but are not limited to, a Read-Only Memory (ROM), a Random-Access Memory (RAM), a flash memory, a removable storage drive, a hard disk drive (HDD), a solid-state memory, a magnetic storage drive, a Programmable Read Only Memory (PROM), an Erasable PROM (EPROM), and/or an Electrically EPROM (EEPROM). Some aspects of the present disclosure are intended to include or otherwise cover any type of the first memory 112 including known, related art, and/or later developed memories. In some aspects of the present disclosure, the device memory 114 may be configured to store blood donation or reception data of the user device 102 corresponding to a user.

The donation console 116 may be configured as a computer-executable application, to be executed by the processing unit 112. The donation console 116 may include suitable logic, instructions, and/or codes for executing various operations and may be controlled by the data processing apparatus 104. The one or more computer executable applications may be stored in the device memory 114. In some aspects of the present disclosure, the user device 102 by way of the donation console 116 may be configured to share blood donation data of the user device 102 corresponding to the user with the plurality of data storage nodes 106 by way of the data processing apparatus 104. In some aspects of the present disclosure, the donation console 116 may include an emergency button (SOS) that may enable a user of the plurality of users to provide an emergency input to the system 100 corresponding to an emergency of donation such that upon reception of the emergency input, the data processing apparatus 104 may be configured to generate an emergency signal to perform one or more operations of the system specific to donation, reception, and/or management of one or more donation samples. In some aspects of the present disclosure, upon generation of the emergency signal, the data processing apparatus 104 may enable a communication of the user with an ambulance in real time.

The communication interface 118 may be configured to enable the user device 102 to communicate with the data processing apparatus 104 and the plurality of data storage nodes 106 (via the data processing apparatus 104) over the communication network 106. Examples of the communication interface 118 may include, but are not limited to, a modem, a network interface such as an Ethernet card, a communication port, and/or a Personal Computer Memory Card International Association (PCMCIA) slot and card, an antenna, a radio frequency (RF) transceiver, one or more amplifiers, a tuner, one or more oscillators, a digital signal processor, a coder-decoder (CODEC) chipset, a subscriber identity module (SIM) card, and a local buffer circuit. It will be apparent to a person of ordinary skill in the art that the communication interface 116 may include any device and/or apparatus capable of providing wireless or wired communications between the user device 102 and the data processing apparatus 104.

In some other aspects of the present disclosure, the system 100 may include a plurality of user devices (not shown) such that each user device of the plurality of user devices may correspond to each user of the plurality of users. The plurality of user devices may be communicatively coupled to the data processing apparatus 104 and the plurality of data storage nodes 106 by way of the communication network 108 such that each user device of the plurality of user devices may be configured have one or more functional components similar to the user device 102, and may perform one or more operations similar to the user device 102.

The data processing apparatus 104 may be a network of computers, a software framework, or a combination thereof, that may provide a generalized approach to create a server implementation. Examples of the data processing apparatus 104 may include, but are not limited to, personal computers, laptops, mini-computers, mainframe computers, any non-transient and tangible machine that can execute a machine-readable code, cloud-based servers, distributed server networks, or a network of computer systems. The data processing apparatus 104 may be realized through various web-based technologies such as, but not limited to, a Java web-framework, a .NET framework, a personal home page (PHP) framework, or any web-application framework. In some aspects of the present disclosure, the data processing apparatus 104 may act as a medium of communication between the user device 102 and the block chain network 106. In other aspects of the present disclosure, the data processing apparatus 104 may be configured to store a backup of data associated with the system 100.

The data processing apparatus 104 may include a processing circuitry 120 and a database 122. The processing circuitry 120 may include suitable logic, instructions, circuitry, interfaces, and/or codes for executing one or more operations of various operations performed by the data processing apparatus 104 for computations and processing related to management of the healthcare donations. The processing circuitry 120 may be configured to host and enable the donation console 116 running on (or installed on) the user device 102 to execute the operations associated with the system 100 by communicating one or more commands and/or instructions over the communication network 108. Examples of the processing circuitry 120 may include, but are not limited to, an ASIC processor, a RISC processor, a CISC processor, a FPGA, and the like.

The database 122 may be configured to store the logic, instructions, circuitry, interfaces, and/or codes of the processing circuitry 120 for executing various operations. In some aspects of the present disclosure, the database 122 may be further configured to store therein, data associated with the plurality of users. Aspects of the present disclosure are intended to include and/or otherwise cover any type of the data associated with the plurality of users. Examples of the database 122 may include but are not limited to, a ROM, a RAM, a flash memory, a removable storage drive, a HDD, a solid-state memory, a magnetic storage drive, a PROM, an EPROM, and/or an EEPROM.

The plurality of data storage nodes 106 may be deployed in a distributed network such as a blockchain or distributed ledger (DLT) employing the plurality of data storage nodes 106 deployed in a mesh topology. Each node of the plurality of data storage nodes 106 may be configured to co-operatively operate for storage of data associated with the plurality of users in a distributed manner. The plurality of data storage nodes 106 may further be configured to perform one or more computational operations based on a set of predefined protocols (such as smart contracts).

The communication network 108 may include suitable logic, circuitry, and interfaces that may be configured to provide several network ports and several communication channels for transmission and reception of data related to operations of various entities (such as the user device 102, and the data processing apparatus 104, and the plurality of data storage nodes 106) of the system 100. Each network port may correspond to a virtual address (or a physical machine address) for transmission and reception of the communication data. For example, the virtual address may be an Internet Protocol Version 4(IPV4 ) (or an IPV6 address) and the physical address may be a Media Access Control (MAC) address. The communication network 108 may be associated with an application layer for implementation of communication protocols based on one or more communication requests from the user device 102, and the data processing apparatus 104, and the plurality of data storage nodes 106. The communication data may be transmitted or received via the communication protocols. Examples of the communication protocols may include, but are not limited to, Hypertext Transfer Protocol (HTTP), File Transfer Protocol (FTP), Simple Mail Transfer Protocol (SMTP), Domain Network System (DNS) protocol, Common Management Interface Protocol (CMIP), Transmission Control Protocol and Internet Protocol (TCP/IP), User Datagram Protocol (UDP), Long Term Evolution (LTE) communication protocols, or any combination thereof. In some aspects of the present disclosure, the communication data may be transmitted or received via at least one communication channel of several communication channels in the communication network 108. The communication channels may include, but are not limited to, a wireless channel, a wired channel, a combination of wireless and wired channel thereof. The wireless or wired channel may be associated with a data standard which may be defined by one of a Local Area Network (LAN), a Personal Area Network (PAN), a Wireless Local Area Network (WLAN), a Wireless Sensor Network (WSN), Wireless Area Network (WAN), Wireless Wide Area Network (WWAN), a metropolitan area network (MAN), a satellite network, the Internet, an optical fiber network, a coaxial cable network, an infrared (IR) network, a radio frequency (RF) network, and a combination thereof. Aspects of the present disclosure are intended to include or otherwise cover any type of communication channel, including known, related art, and/or later developed technologies.

In operation, the data processing apparatus 106, by way of the processing circuitry 120, may be configured to receive a set of user inputs from the plurality of users received by way of the user device 102. The processing circuitry 120 may further be configured to generate a user identifier (UID) for each user of a plurality of users based on a set of user inputs received by the processing circuitry 120. The set of user inputs may be associated with donation of the one or more donation samples. The one or more donation samples may include at least one of, one or more blood units, one or more body organs, or a combination thereof. Upon generation of the UID, the data processing apparatus 104, by way of the processing circuitry 120, may be configured to generate a sample identifier (SID) for each donation sample of the one or more donation samples donated by a first user of the plurality of users. The data processing apparatus 104, by way of the processing circuitry 120, may further be configured to associate the SID of each donation sample with a UID of the first user upon a donation of the donation sample and a UID of a second user of the plurality of users upon a reception of the donation sample. Furthermore, the data processing apparatus 104, by way of the processing circuitry 120 may be configured to generate first and second user profiles for the first and second users, respectively, based on the association of the SID of each donation sample with the UID of the first user. Upon generation of the first and second user profiles, the data processing apparatus 104, by way of the processing circuitry 120, may be configured to distribute the first and second user profiles on the plurality of data storage nodes 106. The data processing apparatus 104, by way of the processing circuitry 120, may be configured to set a priority of the first user for donation of a sample of the one or more donation samples and a priority of the second user for reception of a sample of the one or more donation samples based on the first and second user profiles, respectively.

In some aspects of the present disclosure, the data processing apparatus 106, by way of the processing circuitry 120, may further be configured to associate the SIDs of the at least one donation sample with a UID of the third user that may collect at least one donation sample of the one or more donation sample from the first user and may provide the at least one donation sample to the second user. Further, the data processing apparatus 104, by way of the processing circuitry 120, may be configured to determine a count of donation samples of the at least one donation sample collected or provided by the third user, based on a count of SIDs associated with the UID of the third user. Based on the count of donation samples collected or provided by the third user, the data processing apparatus 104, by way of the processing circuitry 120, may be configured to generate a third donation score for the third user. The data processing apparatus 104, by way of the processing circuitry 120 may further be configured to generate a third status flag for the third user based on the set of health inputs of the first and second users received from the user device 102 and the third donation score. Furthermore, the data processing apparatus 104, by way of the processing circuitry 120 may be configured to generate a third user profile for the third user based on the third status flag, the third UID and the third donation score. Furthermore, the data processing apparatus 104, by way of the processing circuitry 120 may be configured to update the first and second user profiles based on the third user profile. Furthermore, the data processing apparatus 104, by way of the processing circuitry 120 may be configured to distribute the third user profile and the first and second updated user profiles on the plurality of data storage nodes 106. Furthermore, the data processing apparatus 104, by way of the processing circuitry 120, may be configured to set a priority of the third user for at least one of, the donation of one or more donation samples from the first user and the reception of one or more donation samples from the second user based on the third user profile.

In some other aspects of the present disclosure, the data processing apparatus 104, by way of the processing circuitry 120, may be configured to generate a goods identifier (GID) of each health good of the one or more health goods provided by the fourth user that may provide one or more health goods to at least one of, the first through third users, or a combination thereof. The data processing apparatus 120 may further be configured to associate the GIDs of the one or more health goods with a UID of the fourth user. Furthermore, the data processing apparatus 104, by way of the processing circuitry 120, may be configured to determine a first through third count of goods provided by the fourth user to the first through third users based on a count of GIDs associated with the UID of the first through third users, respectively. Furthermore, the data processing apparatus 104, by way of the processing circuitry 120, may be configured to generate a sale score for the fourth user based on first through third count of goods. Furthermore, the data processing apparatus 104, by way of the processing circuitry 120, may be configured to generate a fourth status flag for the fourth user based on the sale score. Furthermore, the data processing apparatus 104, by way of the processing circuitry 120, may be configured to generate a fourth user profile for the fourth user based on the fourth status flag, the fourth UID and the sale score. Furthermore, the data processing apparatus 104, by way of the processing circuitry 120, may be configured to update at least one of, the first through third user profiles based on the fourth user profile. Furthermore, the data processing apparatus 104, by way of the processing circuitry 120 may be configured to distribute the fourth user profile and the at least one of, the first through third updated user profiles on the plurality of data storage nodes 106.

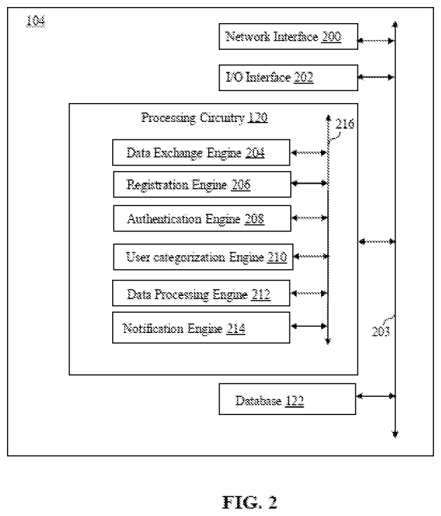

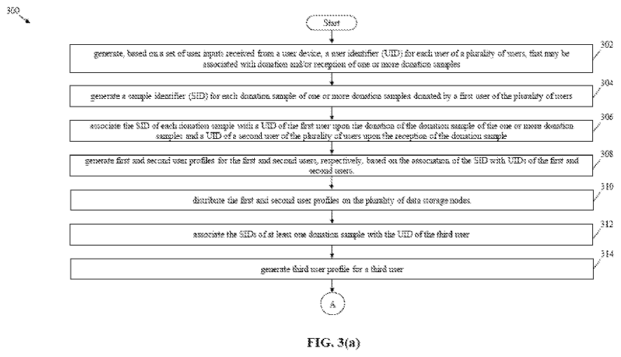

FIG. 2 is a block diagram that illustrates the data processing apparatus 104 of FIG. 1, in accordance with an exemplary aspect of the present disclosure. The data processing apparatus 106 may include the processing circuitry 120 and the Database 122. The data processing apparatus 106 may further include a network interface 200 and an input/output (I/O) interface 202. The processing circuitry 120, the database 122, the network interface 200, and the input/output (I/O) interface 202 may be configured to communicate with each other, by way of a first communication bus 203.

In some aspects of the present disclosure, the processing circuitry 120 may include a data exchange engine 204, a registration engine 206, an authentication engine 208, a user identification engine 210, a data processing engine 212, and a notification engine 214, that may be communicatively coupled to each other, by way of a second communication bus 216. It will be apparent to a person having ordinary skill in the art that the data processing apparatus 106 is for illustrative purposes and not limited to any specific combination of hardware circuitry and/or software.

The data exchange engine 204 may be configured to enable transfer of data from the database to various engines of the processing circuitry 120. The data exchange engine 204 may further be configured to enable a transfer of data and/or instructions from the user device 102 to the data processing apparatus 106. Furthermore, the data exchange engine 204 may be configured to enable transfer of the data and/or instructions between various other engines of the processing circuitry 120.

The registration engine 206 may be configured to enable the user to register into the system 100 by providing registration data through a registration menu (not shown) of the donation console 116 that may be displayed, by way of the user device 102.

In some aspects of the present disclosure, the registration engine 206 may be configured to receive the set of user inputs from the user device 102. In some aspects of the present disclosure, the set of user inputs may include biometric data of the plurality of users such as, but not limited to, fingerprints, iris scans, images, voice samples, and the like associated with the plurality of users. Aspects of the present disclosure are intended to include or otherwise cover any type of biometric data of the plurality of users without deviating from the spirit and scope of the present disclosure.

The authentication engine 208, by way of the data exchange engine 204 may be configured to fetch the registration data of the user and authenticate the registration data of the user. The authentication engine 208, upon successful authentication of the registration data of the user, may be configured to enable the user to log-in or sign up to the system 100. In some aspects of the present disclosure, the authentication engine 208 may enable the user to set the password protection for logging-in to the system 100. In such a scenario, the authentication engine 208 may be configured to verify a password entered by the user for logging-in to the system 100 by comparing the password entered by the user with the set password protection. In some aspects, when the password entered by the user is verified by the authentication engine 208, the authentication engine 208 may enable the user to log-in to the system 100. In some other aspects of the present disclosure, when the password entered by the user is not verified by the authentication engine 208, the authentication engine 208 may generate a signal for the notification engine 224 to generate a login failure notification for the user.

In some other aspects of the present disclosure, the authentication engine 208 may be configured to verify a biometric data of the plurality of users while logging-in to the system 100. For verification of the biometric data, the authentication engine 208 may be configured to compare the biometric data provided by the user while logging-in to the system 100 (or fetched by the system 100) with the biometric data of the plurality of users provided by the plurality of users for registration to the system 100. Upon verification of the biometric data, the authentication engine 208 may enable the user to log-in to the system 100. In some other aspects of the present disclosure, when the biometric data of a user of the plurality of users for logging-in is mismatched by the authentication engine 208, the authentication engine 208 may generate the signal for the notification engine 224 to generate the login failure notification for the user. The verification of each user of the plurality of users (i.e., biometric firewall) before donation of reception (i.e., prior to logging-in to the system 100) may avoid black marketing of healthcare samples.

In some aspects of the present disclosure, for determination of a consent of a user of the plurality of users, the authentication engine 208 may be configured to generate a one-time password (OTP) for the user. The authentication engine 208 may further be configured to generate an OTP transmission signal for transmission of the OTP to the user via one or more mediums such as, but not limited to, a text message, a multimedia message, a Bluetooth message, a CPaaS message, an e-mail, and the like. The authentication engine 208 may further be configured to receive confirm a consent of the user of the plurality of users upon reception of the OTP from the user through the user device 102.

Upon successful login, the user categorization engine 210 may be configured to categorize (or segregate) each user of the plurality of users into one of, the first user (e.g., donor), the second user (e.g., recipient), the third user (e.g., donation authority/hospital), and the fourth user (e.g., health goods provider) based on a set of user selection inputs received from each user of the plurality of users. In some aspects of the present disclosure, based on the categorization of the plurality of users, the user categorization engine 210 may be configured to generate a category label for each user of the plurality of users. In some aspects of the present disclosure, the user categorization engine 210 may further be configured to attach the category label to a UID of each user of the plurality of users.

Upon registration and authentication of a user of the plurality of users, the registration engine 206 may be configured to generate a sample identifier (SID) for each donation sample of the one or more donation samples that may be donated by a first user of the plurality of users. In some aspects of the present disclosure, the one or more donation samples may include at least one of, one or more blood units, one or more body organs, or a combination thereof. In some aspects of the present disclosure, the registration engine 206 may be configured to receive a barcode data associated with each donation sample of the one or more donation samples from the user device 102. In some aspects of the present disclosure, the barcode of a donation sample may include spatiotemporal information of the donation sample such as but not limited to date, time, and location of deposition of the sample, and the like. The registration engine 206 may further be configured to store the barcode data of each donation sample of the one or more donation samples with the associated SID.

In some aspects of the present disclosure, the registration engine 206 may further be configured to generate a goods identifier (GID) for each health good of the one or more health goods provided by the fourth user.

Upon generation of a UID for each user of the plurality of users, a SID for each donation sample of the one or more donation samples, and a GID for each health good of the one or more health goods by the registration engine 206, authentication of each user of the plurality of users by the authentication engine 208, and the categorization of the user of the plurality of users by the user categorization engine 210, the user categorization engine 210 may be configured to generate an initialization signal for the data processing engine 212. In some aspects of the present disclosure, the initialization signal may enable the data processing engine 212 to initialize the processing circuitry 120 to perform one or more operations associated with management of the one or more donation samples between the first and second user.

Upon reception of the initialization signal from the user categorization engine 210, the data processing engine 212 may be configured to determine a validity of each donation sample of the one or more donation samples by processing the associated barcode data (i.e., spatiotemporal data) to identify the date and time of donation. The data processing engine 212 may further be configured to associate the SID of each donation sample with a UID of the first user upon a donation of a donation sample of the one or more donation samples. Furthermore, the data processing engine 212 may be configured to associate a UID of a second user of the plurality of users upon a reception of the donation sample.

In some aspects of the present disclosure, the data processing engine 212 may be configured to determine a life cycle of each donation sample of the one or more donation samples as a time difference between a date and time of donation of the sample by the first user and date and time of the reception of the donation sample by the second user, when the validity of the sample is greater than a pre-defined threshold value (that may be zero).

Based on the association of the SIDs and the UIDs, the data processing engine 212 may be configured to generate first and second user profiles for the first and second users, respectively. In some aspects of the present disclosure, to generate the first and second user profiles, the data processing engine 212 may be configured to determine a count of donation samples donated and received by the first and second users, respectively, based on a count of SIDs associated with the UID of the first and second users, respectively. The data processing engine 212 may further be configured to generate first and second donation scores for the first and second users based on the count of donation samples donated and received by the first and second users, respectively. Furthermore, the data processing engine 212 may be configured to generate, first and second status flag for the first and second users, respectively, based on a set of health inputs of the first and second users (that may be received from the user device 102) and the first and second donation scores. In some aspects of the present disclosure, the first user profile may include the UID of the first user, the first donation score, and the first status flag. The second user profile may include the UID of the second user, the second donation score and the second status flag, respectively.

Upon generation of the first and second user profiles, the data processing engine 212 may be configured to distribute the first and second user profiles on a plurality of data storage nodes 106, where the first and second user profiles may be stored in a distributed network (such as a blockchain network). The data processing engine 212 may further be configured to set priorities of the first and second users for donation and reception of one or more donation samples based on the first and second user profiles, respectively.

In some aspects of the present disclosure, upon generation of the UID of the third user, when the third user receives at least one donation sample from the second user, the data processing engine 212 may be configured to associate a SIDs of the at least one donation sample with the UID of the third user. The data processing engine 212 may further be configured to determine a count of donation samples of the at least one donation sample collected or provided by the third user, based on a count of SIDs associated with the UID of the third user. Furthermore, the data processing engine 212 may generate a third donation score for the third user based on the count of donation samples collected or provided by the third user. Furthermore, the data processing engine 212 may be configured to generate a third status flag for the third user based on a set of health inputs of the first and second users (that may be received from the user device 102) and the third donation score.

In some aspects of the present disclosure, the data processing engine 212 may be configured to identify one or more diseases such as thalassemia, cancer, anemia, diabetes, and the like from a donation sample based on one or more blood parameters of the donation sample (that may be received from the set of health inputs of the first and second users). When a donation sample is detected with one or more diseases, the data processing engine 212 may be configured to generate an alert. The data processing engine 212 may further be configured to associate the one or more diseases with a SID of the donation sample and a UID of the first user associated with the SID to prohibit the first user from donation. In some aspects of the present disclosure, the data processing engine 212 may prioritize a user with one or more diseases for reception of one or more donation samples.

In some aspects of the present disclosure, a donation sample may specifically contain either a component of blood such as white blood cells, red blood cells, and platelets, or whole blood. The data processing engine 212 may further be configured to identify one or more components of the donation sample and may assign a validity of donation to the donation sample based on the identified one or more components of the donation sample. The data processing engine 212 may further be configured to associate the validity of each component of the donation sample with a SID of the donation sample. In some aspects of the present disclosure, the data processing engine 212 may be configured to prioritize a donation sample (with lowest validity) of one or more donation samples such that the donation sample (with lower validity) can be provided at priority to the recipient and wastage of the donation sample can be minimized.

In some aspects of the present disclosure, the data processing engine 212 may generate a temporary prohibit signal for the first user upon donation. The temporary prohibit signal may generate a temporary prohibit flag for a UID of the first user that may prohibit the first user for further donation for a predefined duration (that may be three months for blood donation) to ensure a good health status of the first user.

The data processing engine 212 may further be configured to determine a frequency of donation or reception of each user of the plurality of users. The data processing engine 212 may further be configured to identify one or more frequent first users. Based on the first and second user profiles, the data processing engine 212 may be configured to set a priority of the first and second users for donation and reception of one or more donation samples. In some aspects of the present disclosure, the data processing engine 212 may be configured to determine a frequency of donation or reception of each user of the plurality of users. The data processing engine 212 may further be configured to identify one or more frequent first users and one or more frequent second users based on the frequency of donation and reception of each user of the plurality of users. In some aspects of the present disclosure, the data processing engine 212 may be configured to prioritize the one or more frequent first users (i.e., frequent donors) and the one or more frequent second users (i.e., frequent recipients) for reception of one or more donation samples as and when required. In some aspects of the present disclosure, the data processing engine 212 may further be configured to distribute rewards to the one or more frequent first users (i.e., frequent donors).

In some aspects of the present disclosure, upon generation of the GID for each health good, the data processing engine 212 may be configured to associate the GIDs of the one or more health goods with a UID of the fourth user. The data processing engine 212 may further be configured to determine the first through third count of goods provided by the fourth user to the first through third users based on a count of GIDs associated with the UID of the first through third users, respectively. The fourth user may provide one or more health goods to at least one of, the first through third users, or a combination thereof. The data processing engine 212 may further be configured to generate the sale score for the fourth user based on the first through third count of goods. Furthermore, the data processing engine 212 may be configured to generate the fourth status flag for the fourth user based on the sale score. Furthermore, the data processing engine 212 may be configured to generate the fourth user profile for the fourth user based on the fourth status flag, the fourth UID and the sale score. Furthermore, the data processing engine 212 may be configured to update at least one of, the first through third user profiles based on the fourth user profile. Furthermore, the data processing engine 212 may be configured to distribute the fourth user profile and the at least one of, the first through third updated user profiles on the plurality of data storage nodes 106.

In some aspects of the present disclosure, the data processing engine 212 may be configured to receive an SOS signal from a user of the plurality of users by way of the user device 102. In some aspects of the present disclosure, the SOS signal may include a type of one or more donation samples required by the user. Upon reception of the SOS signal from the user device 102, the data processing engine 212 may be configured to determine a location of the user based on a communication with a location provider (such as Google Maps). The data processing engine 212 may further generate an emergency signal that may include the location of the user and the type of the one or more donation samples required by the user. The data processing engine 212 may further be configured to transmit the emergency signal to the third user and/or a network of ambulances (external to the system 100) such that the user can be reached out for help immediately. In some aspects of the present disclosure, the data processing engine 212 may further be configured to identify one or more nearest ambulances of the network of ambulances to enable the user to determine estimated times of arrival (ETA) of each ambulance of the one or more nearest ambulances such that the user may select an ambulance of the one or more nearest ambulances. The notification engine 214 may be configured to generate one or more notifications for the plurality of users in response to one or more operations performed by the processing circuitry 120.

The database 122 may be configured to store data corresponding to the system 100. In some aspects of the present disclosure, the database 122 may be segregated into one or more repositories that may be configured to store a specific type of data. The database 122 may be configured to temporarily store therein, data associated with the plurality of users (such as the first through fourth user profiles), the various inputs received by the plurality of users (such as the set of health inputs, the set of user inputs, the SOS signal, and the like), data associated with the network of ambulances, and the like, prior to distribution on the plurality of nodes 106 (i.e., blockchain network). The database 122 may further be configured to store therein, a set of instructions for various operations performed by the processing circuitry 120, that may be associated with management of the one or more donation samples.

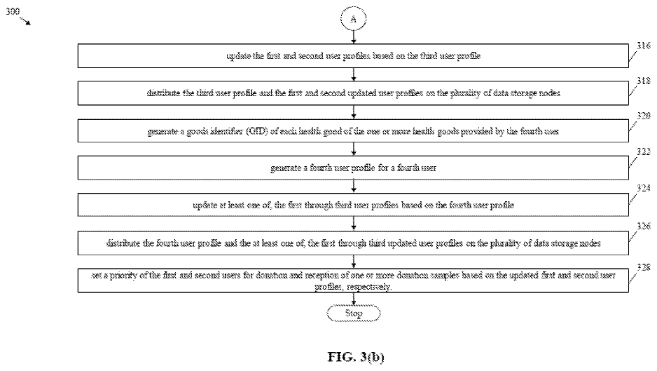

FIG. 3 illustrates a flow chart of a method 300 for the heart rate (HR) determination in accordance with an exemplary aspect of the present disclosure.

At step 302, the data processing apparatus 104 may generate, based on the set of user inputs received from the user device 102, a user identifier (UID) for each user of the plurality of users, that may be associated with donation and/or reception of one or more donation samples.

At step 304, the data processing apparatus 104 may generate a sample identifier (SID) for each donation sample of the one or more donation samples donated by the first user of the plurality of users.

At step 306, the data processing apparatus 104 may associate the SID of each donation sample with a UID of the first user upon the donation of the donation sample of the one or more donation samples and a UID of the second user of the plurality of users upon the reception of the donation sample.

At step 308, the data processing apparatus 104 may generate the first and second user profiles for the first and second users, respectively, based on the association.

In some aspects of the present disclosure, to generate the first and second user profiles, the data processing apparatus 104 may determine the count of donation samples donated and received by the first and second users, respectively, based on the count of SIDs associated with the UID of the first and second users, respectively. The data processing apparatus 104 may further generate the first and second donation scores for the first and second users based on the count of donation samples donated and received by the first and second users, respectively. Furthermore, the data processing apparatus 104 may generate the first and second status flag for the first and second users, respectively, based on the set of health inputs of the first and second users received from the user device 102 and the first and second donation scores. Furthermore, the data processing apparatus 104 may generate the first and second user profiles the first user profile based on the UID of the first user, the first donation score, and the first status flag, and the second user profile comprising the UID of the second user, the second donation score, and the second status flag, respectively.

At step 310, the data processing apparatus 104 may distribute the first and second user profiles on the plurality of data storage nodes 106.

At step 312, the data processing apparatus 104 may associate the SIDs of the at least one donation sample with the UID of the third user.

At step 314, the data processing apparatus 104 may generate the third user profile for the third user.

In some aspects of the present disclosure, to generate the third user profile of the third user, the data processing apparatus 104 may determine the count of donation samples of the at least one donation sample collected or provided by the third user, based on the count of SIDs associated with the UID of the third user. The data processing apparatus 104 may further generate the third donation score for the third user based on the count of donation samples collected or provided by the third user. Furthermore, the data processing apparatus 104 may generate the third status flag for the third user based on the set of health inputs of the first and second users received from the user device 102 and the third donation score. The data processing apparatus 104 may be configured to generate the third user profile of the third user based on the third status flag, the third UID and the third donation score.

At step 316, the data processing apparatus 104 may update the first and second user profiles based on the third user profile.

At step 318, the data processing apparatus 104 may distribute the third user profile and the first and second updated user profiles on the plurality of data storage nodes 106.

At step 320, the data processing apparatus 104 may generate a goods identifier (GID) of each health good of the one or more health goods provided by the fourth user.

At step 322, the data processing apparatus 104 may generate the fourth user profile for the fourth user.

In some aspects of the present disclosure, to generate the fourth user profile, the data processing apparatus 104 may determine the first through third count of goods provided by the fourth user to the first through third users based on the count of GIDs associated with the UID of the first through third users, respectively. The data processing apparatus 104 may further generate the sale score for the fourth user based on the first through third count of goods. Furthermore, the data processing apparatus 104 may generate the fourth user profile for the fourth user based on the fourth status flag, the fourth UID and the sale score.

At step 324, the data processing apparatus 104 may update at least one of, the first through third user profiles based on the fourth user profile.

At step 326, the data processing apparatus 104 may distribute the fourth user profile and the at least one of, the first through third updated user profiles on the plurality of data storage nodes 106.

At step 328, the data processing apparatus 104 may set the priority of the first and second users for donation and reception of one or more donation samples based on the first and second user profiles, respectively.

As mentioned, there is a need for a secure system with automated blood donation management, which is an ongoing effort and demands a need for improvised technical solution that overcomes the aforementioned problems. The present disclosure, therefore provides: the system 100, the data processing apparatus 104, and the method 300 for management of donations. The system 100, by way of the data processing apparatus 104, may generate and share data and metadata of one or more donation samples corresponding to the plurality of users on the plurality of data storage nodes 106. The system 100, by way of the data processing apparatus 104, may help to avoid black marketing of the one or more donation samples by way of the distributed blockchain network. Furthermore, the system 100, by way of the data processing apparatus 106, may generate alerts for the frequent users for donation one or more donation samples. Furthermore, the system 100, by way of the data processing apparatus 104, may store a record of each donation sample in terms of a lifespan to avoid wastage of donated samples and enable proper distribution of donation samples to the receivers by the authorized donation agencies (third users). Furthermore, the system 100, by way of the data processing apparatus 104, may communicate a need of donation samples to the one or more donors (first users). Furthermore, the system 100, by way of the data processing apparatus 104, may further facilitate the one or more donors to communicate with the one or more recipients (second users) and/or the one or more authorized agencies (third users) to share a consent for donation.

The foregoing discussion of the present disclosure has been presented for purposes of illustration and description. It is not intended to limit the present disclosure to the form or forms disclosed herein. In the foregoing Detailed Description, for example, various features of the present disclosure are grouped together in one or more aspects, configurations, or aspects for the purpose of streamlining the disclosure. The features of the aspects, configurations, or aspects may be combined in alternate aspects, configurations, or aspects other than those discussed above. This method of disclosure is not to be interpreted as reflecting an intention the present disclosure requires more features than are expressly recited in each claim. Rather, as the following claims reflect, inventive aspects lie in less than all features of a single foregoing disclosed aspect, configuration, or aspect. Thus, the following claims are hereby incorporated into this Detailed Description, with each claim standing on its own as a separate aspect of the present disclosure.

Moreover, though the description of the present disclosure has included description of one or more aspects, configurations, or aspects and certain variations and modifications, other variations, combinations, and modifications are within the scope of the present disclosure, e.g., as may be within the skill and knowledge of those in the art, after understanding the present disclosure. It is intended to obtain rights which include alternative aspects, configurations, or aspects to the extent permitted, including alternate, interchangeable and/or equivalent structures, functions, ranges, or steps to those claimed, whether or not such alternate, interchangeable and/or equivalent structures, functions, ranges or steps are disclosed herein, and without intending to publicly dedicate any patentable subject matter.

As one skilled in the art will appreciate, the system 100 includes a number of functional blocks in the form of a number of units and/or engines. The functionality of each unit and/or engine goes beyond merely finding one or more computer algorithms to carry out one or more procedures and/or methods in the form of a predefined sequential manner, rather each engine explores adding up and/or obtaining one or more objectives contributing to an overall functionality of the system 100. Each unit and/or engine may not be limited to an algorithmic and/or coded form, rather may be implemented, by way of one or more hardware elements operating together to achieve one or more objectives contributing to the overall functionality of the system 100. Further, as it will be readily apparent to those skilled in the art, all the steps, methods and/or procedures of the system 100 are generic and procedural in nature and are not specific and sequential.

Certain terms are used throughout the following description and claims to refer to particular features or components. As one skilled in the art will appreciate, different persons may refer to the same feature or component by different names. This document does not intend to distinguish between components or features that differ in name but not structure or function. While various aspects of the present disclosure have been illustrated and described, it will be clear that the present disclosure is not limited to these aspects only. Numerous modifications, changes, variations, substitutions, and equivalents will be apparent to those skilled in the art, without departing from the spirit and scope of the present disclosure, as described in the claims.

We claim:

1. A data processing apparatus (104) comprising:
processing circuitry (120) configured to:
generate, based on a set of user inputs received from a user device (102), a user identifier (UID) for each user of a plurality of users, that is associated with a donation of one or more donation samples, wherein the one or more donation samples comprising at least one of, one or more blood units, one or more body organs, or a combination thereof;
generate a sample identifier (SID) for each donation sample of the one or more donation samples donated by a first user of the plurality of users;
associate the SID of each donation sample with (i) a UID of the first user upon a donation of a donation sample of the one or more donation samples and (ii) a UID of a second user of the plurality of users upon a reception of the donation sample;
generate first and second user profiles for the first and second users, respectively, based on the association;
distribute the first and second user profiles on a plurality of data storage nodes (106); and
set a priority of the first and second users for donation and reception of one or more donation samples based on the first and second user profiles, respectively.

2. The data processing apparatus (104) as claimed in claim 1, wherein, to generate the first and second user profiles, the processing circuitry (120) is configured to (i) determine a count of donation samples donated and received by the first and second users, respectively, based on a count of SIDs associated with the UID of the first and second users, respectively, (ii) generate first and second donation scores for the first and second users based on the count of donation samples donated and received by the first and second users, respectively, and (iii) generate, first and second status flag for the first and second users, respectively, based on a set of health inputs of the first and second users received from the user device (102) and the first and second donation scores;
wherein the first user profile comprising a UID of the first user, the first donation score, and the first status flag, and the second user profile comprising a UID of the second user, the second donation score, and the second status flag, respectively.

3. The data processing apparatus (104) as claimed in claim 1, wherein the processing circuitry (120) is further configured to (i) associate the SIDs of the at least one donation sample with a UID of the third user, wherein the third user collects at least one donation sample of the one or more donation sample from the first user and provides the at least one donation sample to the second user, (ii) determine a count of donation samples of the at least one donation sample collected or provided by the third user, based on a count of SIDs associated with the UID of the third user, (iii) generate a third donation score for the third user based on the count of donation samples collected or provided by the third user, (iv) generate a third status flag for the third user based on the set of health inputs of the first and second users received from the user device (102) and the third donation score, (v) generate a third user profile for the third user based on the third status flag, the third UID and the third donation score, (vi) update the first and second user profiles based on the third user profile, (vii) distribute the third user profile and the first and second updated user profiles on the plurality of data storage nodes (106), and (viii) set a priority of the third user for at least one of, the donation of one or more donation samples from the first user and the reception of one or more donation samples from the second user based on the third user profile.

4. The data processing apparatus (104) as claimed in claim 1, wherein the processing circuitry (120) is further configured to (i) generate a goods identifier (GID) of each health good of the one or more health goods provided by a fourth user of the plurality of users, wherein the fourth user provides one or more health goods to at least one of, the first through third users, or a combination thereof, (ii) associate the GIDs of the one or more health goods with a UID of the fourth user, (iii) determine a first through third count of goods provided by the fourth user to the first through third users based on a count of GIDs associated with the UID of the first through third users, respectively, (iv) generate a sale score for the fourth user based on the first through third count of goods, (v) generate a fourth status flag for the fourth user based on the sale score, (vi) generate a fourth user profile for the fourth user based on the fourth status flag, the fourth UID and the sale score, (vii) update at least one of, the first through third user profiles based on the fourth user profile, and (viii) distribute the fourth user profile and the at least one of, the first through third updated user profiles on the plurality of data storage nodes (106).

5. A method (300) comprising:
generating, by way of processing circuitry (120), a user identifier (UID) for each user of a plurality of users, that is associated with a donation of one or more donation samples, based on a set of user inputs received from a user device (102);
generating, by way of the processing circuitry (120), a sample identifier (SID) for each donation sample of the one or more donation samples donated by a first user of the plurality of users;
associating, by way of the processing circuitry (120), the SID of each donation sample with (i) UID of the first user upon a donation of a donation sample of the one or more donation samples and (ii) a UID of a second user of the plurality of users upon a reception of the donation sample;
generating, by way of the processing circuitry (120), first and second user profiles for the first and second users, respectively, based on the association;
distributing, by way of the processing circuitry (120), the first and second user profiles on a plurality of data storage nodes (106); and
setting, by way of the processing circuitry (120), a priority of the first and second users for donation and reception of one or more donation samples based on the first and second user profiles, respectively;
wherein the one or more donation samples comprising at least one of, one or more blood units, one or more body organs, or a combination thereof.

6. The method (300) as claimed in claim 5, wherein, for generating the first and second user profiles, the method (300) comprising (i) determining, by way of the processing circuitry (120), a count of donation samples donated and received by the first and second users, respectively, based on a count of SIDs associated with the UID of the first and second users, respectively, (ii) generating, by way of the processing circuitry (120), first and second donation scores for the first and second users based on the count of donation samples donated and received by the first and second users, respectively, and (iii) generating, by way of the processing circuitry (120), first and second status flag for the first and second users, respectively, based on a set of health inputs of the first and second users received from the user device (102) and the first and second donation scores;
wherein the first user profile comprising a UID of the first user, the first donation score, and the first status flag, and the second user profile comprising a UID of the second user, the second donation score, and the second status flag, respectively.

7. The method (300) as claimed in claim 5, further comprising (i) associating, by way of the processing circuitry (120), the SIDs of the at least one donation sample with a UID of the third user of the plurality of users, wherein the third user collects at least one donation sample of the one or more donation sample from the first user and provides the at least one donation sample to the second user, (ii) determining, by way of the processing circuitry (120), a count of donation samples of the at least one donation sample collected or provided by the third user, based on a count of SIDs associated with the UID of the third user, (iii) generating, by way of the processing circuitry (120), a third donation score for the third user based on the count of donation samples collected or provided by the third user, (iv) generating, by way of the processing circuitry (120), a third status flag for the third user based on the set of health inputs of the first and second users received from the user device (102) and the third donation score, (v) generating, by way of the processing circuitry (120), a third user profile for the third user based on the third status flag, the third UID and the third donation score, (vi) updating, by way of the processing circuitry (120), the first and second user profiles based on the third user profile, (vii) distributing, by way of the processing circuitry (120), the third user profile and the first and second updated user profiles on the plurality of data storage nodes (106), and (viii) setting, by way of the processing circuitry (120), a priority of the third user for at least one of, the donation of one or more donation samples from the first user and the reception of one or more donation samples from the second user based on the third user profile.

8. The method (300) as claimed in claim 5, further comprising (i) generating, by way of the processing circuitry (120), a goods identifier (GID) of each health good of the one or more health goods provided by the fourth user, wherein the fourth user provides one or more health goods to at least one of, the first through third users, or a combination thereof, (ii) associating, by way of the processing circuitry (120), the GIDs of the one or more health goods with a UID of the fourth user, (iii) determining, by way of the processing circuitry (120), a first through third count of goods provided by the fourth user to the first through third users based on a count of GIDs associated with the UID of the first through third users, respectively, (iv) generating, by way of the processing circuitry (120), a sale score for the fourth user based on the first through third count of goods, (v) generating, by way of the processing circuitry (120), a fourth status flag for the fourth user based on the sale score, (vi) generate a fourth user profile for the fourth user based on the fourth status flag, the fourth UID and the sale score, (vii) updating, by way of the processing circuitry (120), at least one of, the first through third user profiles based on the fourth user profile, and (viii) distributing, by way of the processing circuitry (120), the fourth user profile and the at least one of, the first through third updated user profiles on the plurality of data storage nodes (106).
